(19) United States
(12) Patent Application Publication
(10) Pub. No.: US 2026/0020982 A1
(43) Pub. Date: Jan. 22, 2026

(54) ADJUSTABLE SHUNTS WITH SHAPE MEMORY ACTUATORS AND ASSOCIATED SYSTEMS AND METHODS

(71) Applicant: Shitamed Holdings, LLC, Campbell, CA (US)

(72) Inventor: Eric Schultz, Los Altos, CA (US)

(21) Appl. No.: 18/997,775

(22) PCT Filed: Jul. 27, 2023
(86) PCT No.: PCT/US2023/071106

§ 371 Date: Jan. 22, 2025

Related U.S. Application Data
(60) Provisional application No. 63/392,695, filed on Jul. 27, 2022.
(60) Provisional application No. 63/486,436, filed on Feb. 22, 2023.

(51) Int. Cl. A61F 9/007

(52) U.S. Cl. CPC A61F 9/00781; A61F 2210/0014; A61F 2230/0054; A61F 2250/0013

(57) ABSTRACT

The present technology provides adjustable shunting systems with shape memory actuators. The shape memory actuators can be configured to selectively control the flow of fluid through the shunting system. For example, the shape memory actuators can include an anchor element, a gating element, and first and second actuation elements that extend between the anchor element and the gating element. The first actuation element can be selectively and independently actuated to rotate, pivot, or otherwise move the gating element in a first direction. The second actuation element can also be selectively and independently actuated to rotate, pivot, or otherwise move the gating element in a second direction opposite the first direction. When the shape memory actuator is coupled to the shunting system, the gating element can be positioned to moveably interface with a port or channel that permits fluid to flow into and/or through the shunting system.

### CROSS-REFERENCE TO RELATED APPLICATION(S)

The present application claims priority to U.S. Provisional Patent Application No. 63/392,695, filed Jul. 27, 2022, and U.S. Provisional Patent Application No. 63/486,436, filed Feb. 22, 2023, the disclosures of which are incorporated by reference herein in their entireties.

### TECHNICAL FIELD

The present technology generally relates to implantable medical devices and, in particular, to adjustable shunts for controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma surgery devices or “MIGS” devices) have shown clinical benefit; however, there is a need for improved shunting systems, systems for delivering such shunting systems, and techniques for addressing elevated intraocular pressure and risks associated with glaucoma. For example, there is a need for shunting systems capable of adjusting the therapy provided to meet the patient's individual and variable needs and/or account for changes in flow-related characteristics, including the flow rate between the two fluidly connected bodies.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A is an isometric view of a shunting system configured in accordance with select embodiments of the present technology.

FIG. 1B is a top view of the shunting system shown in FIG. 1A.

FIG. 1C is an enlarged view of a portion of the shunting system shown in FIGS. 1A and 1B.

FIG. 2A illustrates an actuator of the shunting system shown in FIGS. 1A-1C in an as-manufactured state and configured in accordance with select embodiments of the present technology.

FIG. 2B illustrates the actuator of FIG. 2B loaded on a priming element of the shunting system shown in FIGS. 1A-1C and configured to in accordance with select embodiments of the present technology.

FIG. 2C illustrates the actuator of FIGS. 2A and 2B in a first position following actuation of a first actuation element in accordance with select embodiments of the present technology.

FIG. 2D illustrates the actuator of FIGS. 2A and 2B in a second position following actuation of a second actuation element in accordance with select embodiments of the present technology.

FIG. 3 illustrates another actuator that can used with the shunting system shown in FIGS. 1A-1C and configured in accordance with select embodiments of the present technology.

FIGS. 4A and 4B illustrate an actuator assembly having a first actuator and a second actuator and configured in accordance with select embodiments of the present technology

FIGS. 5A and 5B illustrate another actuator assembly having a first actuator and a second actuator and configured in accordance with select embodiments of the present technology.

FIG. 6A is a perspective view of another adjustable shunting system configured in accordance with select embodiments of the present technology.

FIG. 6B is an exploded view of the adjustable shunting system of FIG. 6A.

### DETAILED DESCRIPTION

The present technology is generally directed to adjustable shunting systems with shape memory actuators. The shape memory actuators can be configured to selectively control the flow of fluid through the shunting system. For example, in some embodiments the shape memory actuators include an anchor element, a gating element, and first and second actuation elements that extend between the anchor element and the gating element. The first actuation element can be selectively and independently actuated to pivot, rotate, or otherwise move the gating element in a first direction. The second actuation element can also be selectively and independently actuated to pivot, rotate, or otherwise move the gating element in a second direction opposite the first direction. When the shape memory actuator is coupled to the shunting system, the gating element can be positioned to moveably interface with a port or channel that permits fluid to flow into and/or through the shunting system. Accordingly, the gating element can be selectively moved, via actuation of the first actuation element and the second actuation element, between at least a first position that blocks or generally blocks fluid flow through the port or channel and a second position that permits or generally permits unimpeded fluid flow through the port or channel.

As described in detail throughout this description, the shape memory actuators described herein are expected to provide several advantageous features. For example, the shape memory actuators described herein can be relatively compact (e.g., have narrow width, etc.) while still providing rotational movement (as opposed to linear-translational movement) of the gating element. This is expected to be beneficial because smaller actuators result in relatively smaller shunting systems, which may reduce certain side effects. Moreover, in some embodiments, the shape memory actuators described herein can also have a reduced or minimized distance between the “pivot point” around which the gating element pivots and the portion of the gating element that interfaces with the port. For example, the gating element can interface with a priming feature to define a pivot region about which the gating element pivots/rotates to selectively control the flow of fluid through the port. As described below, this is expected to be advantageous because it may provide more controllable and repeatable movement of the gating element during operation. Of course, the shape memory actuators and shunting systems described herein may provide additional advantages not explicitly recognized herein.

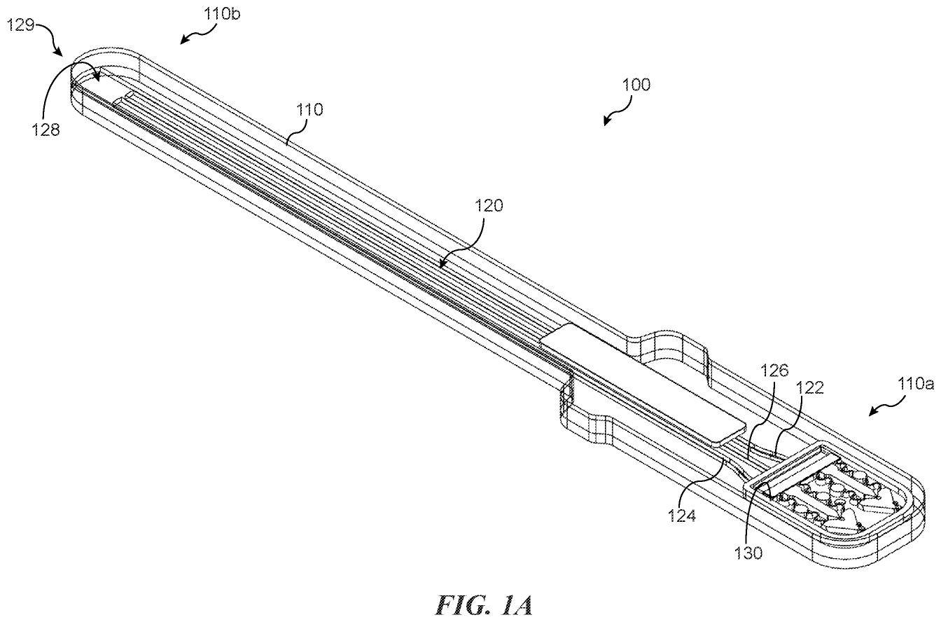

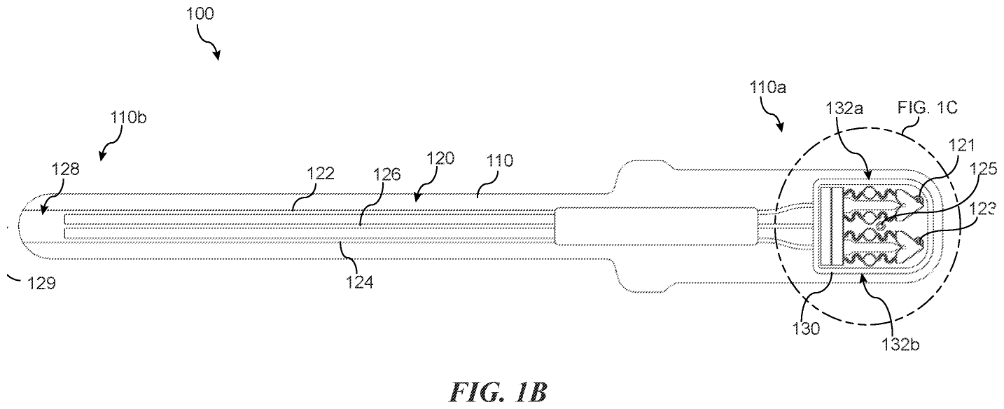

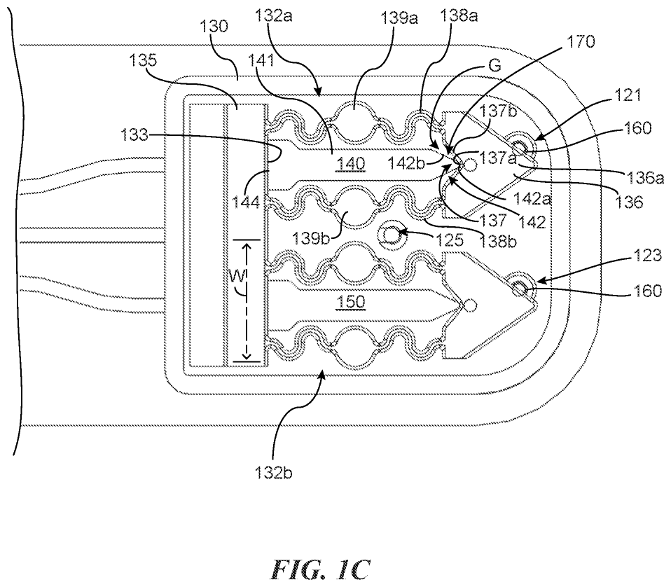

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and claims but are not described in detail with respect to FIGS. 1A-6B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye (including the posterior chamber), or, more generally, from and/or between a first body region and a second body region. Moreover, while the certain embodiments herein are described in the context of glaucoma treatment. any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A-1C illustrate a shunting system 100 (“the system 100”) configured in accordance with select embodiments of the present technology. More specifically, FIG. 1A is an isometric view of the system 100, FIG. 1B is a top-down view of the system 100, and FIG. 1C is an enlarged view of a portion of the system 100 taken along the region identified as “FIG. 1C” in FIG. 1B. As described in greater detail below, the system 100 is configured to provide a titratable therapy for draining fluid from a first body region to a second body region when implanted in a patient, such as to drain aqueous from an anterior chamber of a patient's eye to aid in the treatment of glaucoma.

Referring collectively to FIGS. 1A and 1B, the system 100 includes an elongated housing or shunting element 110 (which can also be referred to as a casing, membrane, shell, shunt body, or the like) and a flow control assembly or cartridge 130. In some embodiments, the shunting element 110 is composed of an elastic or flexible biocompatible material (e.g., silicone, etc.). The shunting element 110 extends between a first end region 110a and a second end region 110b and is configured to facilitate the drainage/shunting of fluid therebetween. For example, as best shown in FIG. 1B, the first end region 110a can include a first port 121 (e.g., a first fluid inlet), a second port 123 (e.g., a second fluid inlet), and a third port 125 (e.g., a third fluid inlet). The second end region 110b includes a fourth port 129 (e.g., an outflow port).

The shunting element 110 can include a fluid resistor network 120 extending therethrough for draining/shunting fluid between the first end region 110a and the second end region 110b. The fluid resistor network 120 can include a plurality of discrete and/or interconnected channels or channel segments. For example, in the illustrated embodiment, the fluid resistor network 120 includes a first channel 122, a second channel 124, and a third channel 126. The first channel 122 is fluidly coupled to (and therefore configured to receive fluid via) the first port 121, the second channel 124 is fluidly coupled to (and therefore configured to receive fluid via) the second port 123, and the third channel 126 is fluidly coupled to (and therefore configured to receive fluid via) the third port 125. Each of the first channel 122, the second channel 124, and the third channel 126 terminate at a common lumen 128, which directs fluid to the fourth port 129. That is, fluid flowing through the fluid resistor network 120 (i) enters the fluid resistor network via one or more of the first port 121, the second port 123, or the third port 125, (ii) flows through the corresponding first channel 122, the second channel 124, or the third channel 126, respectively, (iii) flows into the common lumen 128, and (iv) exits the fluid resistor network 120 via the fourth port 129. As described below, the fluid resistor network 120 can be selectively gated such that fluid can simultaneously flow through all three ports, two of the three ports, or just a single port at anfy given time. In some embodiments, the direction of flow can be reversed (e.g., the system 100 can be configured to shunt fluid from the second end region 110b toward the first end region 110a). Although the fluid resistor network 120 is shown as having three parallel channels, one skilled in the art will appreciate that the fluid resistor network 120 can have more or fewer channels, including one, two, four, five, six, or more channels. Likewise, the fluid resistor network 120 can include channels and/or channel segments arranged in other patterns (e.g., combinations of channels arranged in series or in parallel) different than that illustrated in FIGS. 1A and 1B. The shunting element 110 can also have more or fewer ports, depending on the configuration of the fluid resistor network 120.

The flow control cartridge 130 can include one or more actuators configured to selectively control the flow of fluid through one or more portions of the fluid resistor network 120. For example, the illustrated flow control cartridge 130 includes two actuators: a first actuator 132a configured to selectively control the flow of fluid through the first port 121, and a second actuator 132b configured to selectively control the flow of fluid through the second port 123. In some embodiments, the first actuator 132a can be configured to selectively control the flow of fluid through one or more apertures or ports fluidically between the first port 121 and the first channel 122, in addition to or instead of controlling flow through the first port 121 itself. Likewise, in some embodiments the second actuator 132b can be configured to selectively control the flow of fluid through one or more apertures or ports fluidically between the second port 123 and the second channel 124, in addition to or instead of controlling flow through the second port 123 itself.

FIG. 1C is an enlarged view of a portion of the system 100 including the flow control cartridge 130 (taken along the region identified as “FIG. 1C” in FIG. 1B). As best seen in FIG. 1C, the system 100 includes one or more fluid inlets or apertures 160 (two are shown) for permitting fluid to flow into the system 100. In the illustrated embodiment, the fluid inlets 160 are positioned above and axially aligned with the corresponding first port 121 and second port 123. In other embodiments, however, the fluid inlets 160 may have a different arrangement relative to the first and second ports 121 and 123. In the illustrated embodiment, the fluid inlets 160 have a smaller diameter than the first and second ports 121 and 123. In other embodiments, however, the fluid inlets 160 may have the same diameter or have a larger diameter than the first and second ports 121 and 123.

After permitting fluid into the system 100 via the fluid inlets 160, and as set forth above, the flow control cartridge 130 includes a first actuator 132a for selectively controlling the flow of fluid through the first port 121 and/or otherwise selectively controlling the flow of fluid into the first channel 122, and a second actuator 132b for selectively controlling the flow of fluid through the second port 123 and/or otherwise selectively controlling the flow of fluid into the second channel 124. For purposes of brevity, the first actuator 132a and its components are described in detail below. One skilled in the art will appreciate that the second actuator 132b can have the same or substantially the same components, and operate in the same or substantially the same way, as the first actuator 132a.

The first actuator 132a includes a gating element 136, an anchor element 135, a first actuation element 138a, and a second actuation element 138b. As described in detail below, the gating element 136, the anchor element 135, the first actuation element 138a, and the second actuation element 138b can form a single, contiguous component. For example, the first actuator 132a can be laser cut from a sheet or tube of material, such as a sheet or tube of Nitinol or other suitable material.

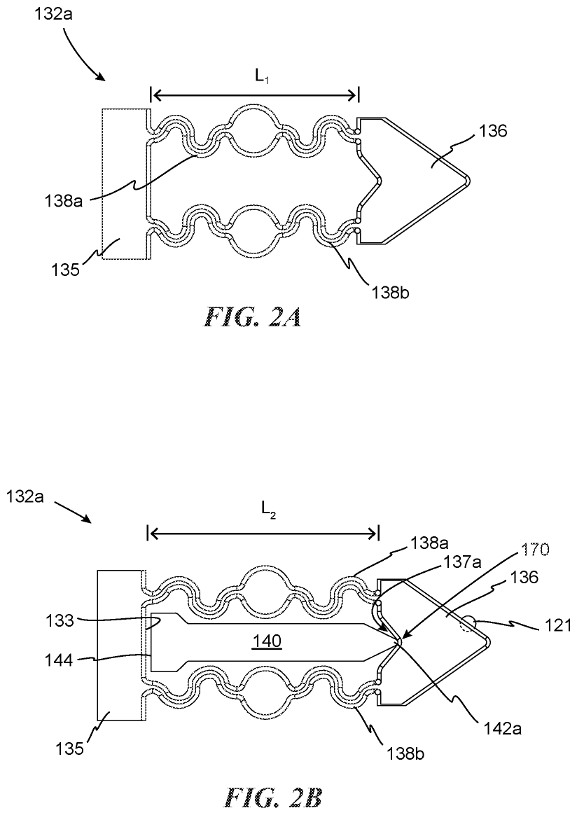

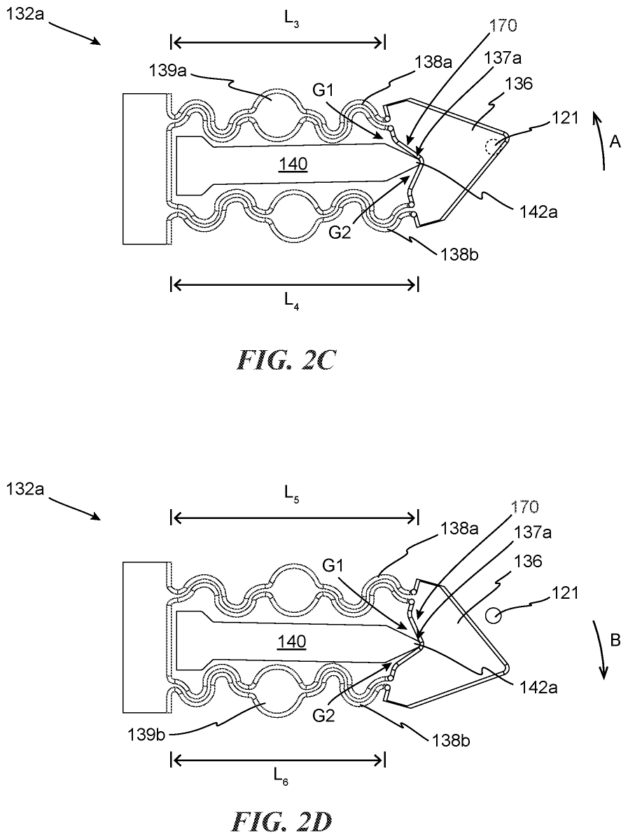

The gating element 136 can be sized and shaped to selectively interface with the first port 121 to control the flow of fluid therethrough. In the illustrated embodiment, for example, the gating element 136 has a general “V” shape, with a blocking portion 136a (e.g., a body portion or an end portion) configured to interface with the port 121, and a recess or valley 137 defined by the gating element 136 configured to act as a pivot region 170. As described in greater detail below, the pivot region 170 includes a pivot point about which the gating element 136 pivots/rotates to selectively control the flow of fluid through the first port 121 and second port 123. For example, as described in greater detail with reference to FIGS. 2A-2D, the gating element 136 can be moveable between at least a first position in which the gating element 136 imparts a first (e.g., high) resistance to fluid flowing through the first port 121 (e.g., the gating element 136 blocks at least 80% of, at least 90% of, at least 95% of, or all of the first port 121), and a second position in which the gating element 136 imparts a second (e.g., low, about zero, or zero) resistance to fluid flowing through the first port 121 (e.g., the gating element 136 blocks less than 20%, less than 10%, less than 5%, and/or none of the first port 121). In FIG. 1C, the gating element 136 is illustrated in an intermediate position between the first position and the second position. As described in detail with reference to FIGS. 2A and 2B, the intermediate position represents the configuration of the first actuator 132a after it has been loaded onto the system 100 but before it has been actuated.

The first actuation element 138a and the second actuation element 138b extend between the gating element 136 and the anchor element 135. That is, the anchor element 135 is coupled to one end of the first actuation element 138a and the second actuation element 138b, and the gating element 136 is coupled to the opposite end of the first actuation element 138a and the second actuation element 138b. This configuration is expected to be advantageous because it results in a relatively compact actuator. For example, relative to shape-memory actuators that have a gating element extending between and generally parallel to the actuation elements, the present shape memory actuators can have a reduced width W (shown on the second actuator 132b simply for clarity). In some embodiments, the width W can be less than about 1 mm, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, less than about 40 microns, less than about 30 microns, or less than about 25 microns. In turn, relative compact actuators are beneficial because they can reduce the overall footprint of the system 100, which may reduce side effects (e.g., tissue irritation, vision blockage, etc.).

As described below with reference to FIGS. 2A-2D, the first actuation element 138a can be selectively actuated to move the gating element 136 to and/or toward the first position that imparts a high resistance through the first port 121, while the second actuation element 138b can be selectively actuated to move the gating element 136 to and/or toward the second position that imparts low or no resistance through the first port 121. As used herein, the term “selectively” when used in the context of actuating an actuation element refers to the ability to actuate one of the first actuation element 138a or the second actuation element 138b on demand, without actuating the other of the first actuation element 138a or the second actuation element 138b. For example, in some embodiments the first actuation element 138a and the second actuation element 138b are activated via application of heat (e.g., via an energy source external to the patient, such as via laser energy delivered from a source external to the patient). In such embodiments, the first actuation element 138a can be selectively actuated by heating the first actuation element 138a without substantially heating the second actuation element 138b. Likewise, the second actuation element 138b can be selectively actuated by heating the second actuation element 138b without substantially heating the first actuation element 138a. Additional details regarding the actuation of the first actuation element 138a and the second actuation element 138b are described with reference to FIGS. 2A-2D.

As set forth above, the first actuator 132a can be composed of a shape memory material or alloy such as Nitinol. Accordingly, portions of the first actuator 132a can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In particular, the first actuation element 138a and the second actuation element 138b can be independently transitionable between the first material state and the second material state. In the first material state, the first actuation element 138a and the second actuation element 138b may have reduced (e.g., relatively less stiff) mechanical properties that cause the first actuation element 138a and the second actuation element 138b to be more easily deformable (e.g., compressible, expandable, etc.), relative to when the first actuation element 138a and the second actuation element 138b are in the second material state. In the second material state, the first actuation element 138a and the second actuation element 138b may have increased (e.g., relatively more stiff) mechanical properties, relative to when the first actuation element 138a and the second actuation element 138b are in the first material state, causing an increased preference toward a specific preferred geometry (e.g., original geometry, manufactured or fabricated geometry, heat set geometry, etc.). The first actuation element 138a and the second actuation element 138b can be selectively and independently transitioned between the first material state and the second material state by heating them above a transition temperature (e.g., above an austenite finish (Af) temperature, which is generally greater than body temperature). In some embodiments, the first actuation element 138a and the second actuation element 138b can be heated above the transition temperature using non-invasive energy, such as laser energy from a source external to the patient. As described below with reference to FIGS. 2A-2D, if the first actuation element 138a or the second actuation element 138b is deformed relative to its preferred geometry when heated above the transition temperature, the first actuation element 138a or the second actuation element 138b will move to and/or toward its preferred geometry. As also described below, this movement of the first and second actuation elements 138a-b causes a corresponding movement of the gating element 136.

In some embodiments, the first actuation element 138a can further include a first target 139a coupled to and/or in-line with the first actuation element 138a. The first target 139a can be integral with and therefore composed of the same material as the first actuation element 138a. Accordingly, the first target 139a can be thermally coupled to the first actuation element 138a that that energy (e.g., laser energy) received at the first target 139a can dissipate through the first actuation element 138a in the form of heat. The first target 139a can therefore be selectively targeted with non-invasive energy to heat, and therefore actuate, the first actuation element 138a. The second actuation element 138b can include a second target 139b that is the same or substantially the same as the first target 139a, except that non-invasive energy received at the second target 139b causes heat to dissipate through the second actuation element 138b. Although the first target 139a and the second target 139b are shown as positioned generally centrally along a length of the first actuation element 138a and the second actuation element 138b, respectively, in other embodiments the first target 139a and/or the second target 139b can be coupled to the first actuation element 138a and the second actuation element 138b at other suitable positions, such as at an end region of the first actuation element 138a and the second actuation element 138b. Without being bound by theory, the increased surface area of the first target 139a relative to the first actuation element 138a and of the second target 139b relative to the second actuation element 138b is expected to increase the ease and consistency by which the energy can be delivered to the first actuation element 138a and the second actuation element 138b.

The flow control cartridge 130 can also include an actuator priming element 140 (“the priming element 140”), which can also be referred to as an actuator loading element, an actuator bracing element, an actuator retaining element, or an actuator receiving element. The priming element 140 has an elongated body 141 extending between a first end portion 142 and a second end portion 144. As shown, the first end portion 142 can be inwardly tapered or angled such that a width of the first end portion 142 decreases from the body 141 toward a tip or apex 142a (e.g., pivot). In particular, the first end portion 142 is tapered such that the first end portion 142 is narrower than the recess 137 formed in the gating element 136. As a result, the first end portion 142 can extend into the recess 137 such that the tip 142a of the first end portion 142 contacts the gating element 136 at a “bottom” or deepest portion 137a of the recess 137. Moreover, because the first end portion 142 is narrower than the recess 137, side walls 137b (only one is labeled in FIG. 1C for clarity) of the gating element 136 that extend from the deepest portion 137a to form the recess 137 are spaced apart from corresponding side walls 142b (only one is labeled in FIG. 1C for clarity) of the first end portion 142 of the priming element 140. As a result, a gap G is formed between the side walls 137b of the recess 137 and the side walls 142b of the priming element 140. As described in detail below with reference to FIGS. 2A-2D, this enables the tip 142a to act as a pivot about which the gating element 136 can pivot, rotate, or otherwise move. The second end portion 144 of the priming element 140 can be configured to abut an anchoring surface 133 of the anchor element 135. The flow control cartridge 130 can further include a second actuator priming element 150 configured to interface with the second actuator 132b.

In some embodiments, the priming element 140 may be composed of a material that is generally stiffer than the first actuator 132a, such that the priming element 140 does not deform during operation of the first actuator 132a. For example, in some embodiments the priming element 140 is composed of plastic, glass, steel, etc. The priming element 140 can also have other configurations different than the configuration illustrated in FIG. 1C. For example, in some embodiments the priming element 140 may have two discrete components form the “first end portion” and the “second end portion.” In such embodiments, the body 141 can be omitted such that there is a gap between the first end portion 142 and the second end portion 144. Regardless of the configuration of the priming element 140, the second actuator priming element 150 can be the same as or generally similar to the priming element 140, and so the foregoing description of the priming element 140 applies equally to the second priming element 150.

FIGS. 2A-2D illustrate additional details of the operation of the first actuator 132a in accordance with select embodiments of the present technology. A number of components/features of the flow control cartridge 130 and the system 100 described above are omitted from FIGS. 2A-2D for purposes of illustration and clarity. Referring first to FIG. 2A, the first actuator 132a is shown in its preferred geometry. That is, the configuration shown in FIG. 2A represents the first actuator 132a in its “as-fabricated” or “manufactured” state, before it has been deformed. In its preferred geometry, the first actuation element 138a and the second actuation element 138b have a first length L₁. Although shown has having the same or generally the same first length L₁, in some embodiments the first actuation element 138a and the second actuation element 138b can have different lengths in the preferred geometry.

FIG. 2B illustrates the first actuator 132a “loaded” onto the priming element 140 and in the intermediate position between the first position and the second position. As shown, to load the first actuator 132a onto the priming element 140, the first actuator 132a must be stretched or otherwise tensioned such that (1) the deepest portion 137a of the gating element 136 can fit over the tip 142a of the priming element, and (2) the anchoring surface 133 of the anchor element 135 can abut the second end portion 144 of the priming element 140. This causes the first actuation element 138a and the second actuation element 138b to “deform” relative to their preferred geometries. More specifically, the first actuation element 138a and the second actuation element 138b are both lengthened relative to their preferred geometry to a second length L₂. The second length L₂ can be between about 2% and about 20% greater than, or between about 5% and about 15% greater than, the first length, although in other embodiments the relationship between the second length L₂, and the first length Li can be outside the foregoing ranges. The deformation of the first actuation element 138a and the second actuation element 138b tensions or loads the first actuation element 138a and the second actuation element 138b, enabling them to drive movement of the gating element 136 when actuated, as described below with respect to FIGS. 2C and 2D.

FIG. 2C illustrates the first actuator 132a in the first position following actuation of the first actuation element 138a, and FIG. 2D illustrates the first actuator 132a in the second position following actuation of the second actuation element 138b. To move from the intermediate position (FIG. 2B) or from the second position (FIG. 2D) to and/or toward the first position (FIG. 2C), the first actuation element 138a is actuated. More specifically, the first actuation element 138a is heated above its transition temperature to transition from the first material state to and/or toward the second material state, as described previously. In some embodiments, as noted previously, this is accomplished by directing non-invasive energy (e.g., from a laser positioned external to the patient) at the first target 139a. The energy received at the first target 139a dissipates through the first actuation element 138a in the form of heat as described above. Because the first actuation element 138a is deformed relative to its preferred geometry (FIG. 2A) in both the intermediate position (FIG. 2B) and the second position (FIG. 2D), heating the first actuation element 138a above its transition temperature causes the first actuation element 138a to move toward its preferred geometry. That is, the first actuation element 138a contracts or shortens to a third length L₃. In some embodiments, the third length L₃ is the same or generally the same as the first length L₁, which represents the length of the first actuation element 138a in its preferred geometry without external biasing forces. However, in other embodiments, the third length L₃ may be slightly greater than the first length L₁ (although less than the second length L₂) by virtue of forces imparted on the first actuation element 138a by the second actuation element 138b being further lengthened relative to its preferred geometry. For example, as shown in FIG. 2C, following actuation of the first actuation element 138a, the second actuation element 138b is further deformed (e.g., lengthened) relative to its preferred geometry to a fourth length L₄. The fourth length La is greater than each of the first length L₁ (FIG. 2A), the second length L₂ (FIG. 2B), and the third length L₃ (FIG. 2C). Even through the second actuation element 138b remains in the first material state, it may nevertheless impart a slight biasing force against the first actuation element 138a that prevents the first actuation element 138a from contracting fully to the first length L₁.

Actuating the first actuation element 138a causes a corresponding movement in the gating element 136. For example, as shown in FIG. 2C, actuating the first actuation element 138a causes the gating element 136 to pivot in a direction indicated by the arrow A. More specifically. the gating element 136 pivots about the tip 142a of the priming element 140, which is enabled by virtue of the gap G between the side walls 142b of the first end portion 142 of the priming element 140 and the side walls 137b that form the recess 137 of the gating element 136 (FIG. 1C). This decreases a first gap G₁ between the priming element 140 and the gating element 136 and increases a second gap G₂ between the priming element 140 and the gating element 136. This also moves the gating element 136 to the first position in which it confers the high first fluid resistance through the first port 121. For example, in the first position, the gating element 136 can block or substantially block fluid from flowing through the port 121.

To move from the intermediate position (FIG. 2B) or from the first position (FIG. 2C) to and/or toward the second position (FIG. 2D), the second actuation element 138b is actuated. More specifically, the second actuation element 138b is heated above its transition temperature to transition from the first material state to and/or toward the second material state, as described previously. In some embodiments, as also described above, this is accomplished by directing non-invasive energy (e.g., from a laser positioned external to the patient) at the second target 139b. The energy received at the second target 139b dissipates through the second actuation element 138b in the form of heat, as described above. Because the second actuation element 138b is deformed relative to its preferred geometry (FIG. 2A) in both the intermediate position (FIG. 2B) and the first position (FIG. 2C), heating the second actuation element 138b above its transition temperature causes the second actuation element 138b to move toward its preferred geometry. That is, the second actuation element 138b contracts or shortens to a fifth length L₅. In some embodiments, the fifth length L₅ is the same or generally the same as the first length L₁, which represents the length of the second actuation element 138b in its preferred geometry without external biasing forces. However, in other embodiments, the fifth length L₅ may be slightly greater than the first length L₁ (although less than the second length L₂) by virtue of forces imparted on the second actuation element 138b by the first actuation element 138a being further lengthened relative to its preferred geometry. For example, as shown in FIG. 2D, following actuation of the second actuation element 138b, the first actuation element 138a is further deformed (e.g., lengthened) relative to its preferred geometry to a sixth length L₆. The sixth length L₆ is greater than each of the first length L₁ (FIG. 2A), the second length L₂ (FIG. 2B), the third length L₃ (FIG. 2C), and the fifth length L₅. In some embodiments, the sixth length L₆ can be the same or generally the same as the fourth length L₄ (FIG. 2C).

Actuating the second actuation element 138b also causes a corresponding movement in the gating element 136. For example, as shown in FIG. 2D, actuating the second actuation element 138b causes the gating element 136 to pivot in a direction indicated by the arrow B. More specifically, the gating element 136 pivots about the tip 142a of the priming element 140, as described above in the context of actuating the first actuation element 138a. However, the rotation induced by actuating the second actuation element 138b is in the opposite direction to the rotation induced by actuating the first actuation element 138a. Accordingly, actuating the second actuation element 138b increase the first gap G₁ between the priming element 140 and the gating element 136 and decreases the second gap G₂ between the priming element 140 and the gating element 136. This also moves the gating element 136 to the second position in which it confers the low or no fluid resistance through the first port 121. For example, in the second position, the gating element 136 does not block or substantially block fluid from flowing through the port 121.

As described above, the first actuation element 138a and the second actuation element 138b generally act in opposition. As the first actuation element 138a moves toward its preferred geometry, the second actuation element 138b is further deformed relative to its preferred geometry. Likewise, as the second actuation element 138b moves toward its preferred geometry, the first actuation element 138a is further deformed relative to its preferred geometry. Additional details of shape memory actuation elements are described in U.S. Patent Application Publication Nos. US 2020/0229982 and US 2021/0251806, the disclosures of which are both incorporated by reference herein in their entireties and for all purposes.

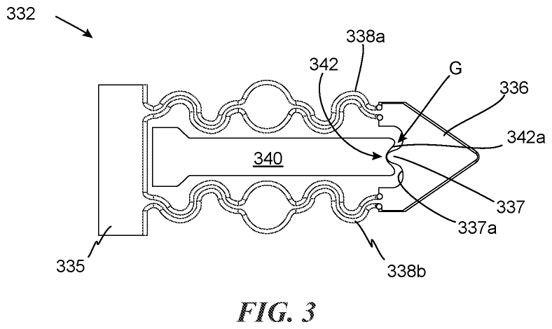

The actuators and adjustable shunts can have other suitable configurations. For example, FIG. 3 illustrates another actuator 332 and actuator priming element 340 configured in accordance with select embodiments of the present technology. The actuator 332 and the priming element 340 can be generally similar to the actuator 132a and the priming element 140 described previously with reference to FIGS. 1A-2D. For example, the actuator 332 can include a gating element 336, an anchor element 335, a first actuation element 338a, and a second actuation element 338b. The actuator 332 can be loaded onto the actuator priming element 340, and the first actuation element 338a and the second actuation element 338b can then be selectively actuated to rotate, pivot, or otherwise move the gating element 336, as described in detail above with reference to FIGS. 2A-2D. Accordingly, the following description focuses on the features of the actuator 332 and the priming element 340 that substantially differ from the actuator 132a and the priming element 140 described previously, with the understanding that the description of the actuator 132a and the priming element 140 can apply to the actuator 332 and the priming element 340, unless the context clearly dictates otherwise.

As shown in FIG. 3, the gating element 336 of the actuator 332 includes a projection or nub 337 configured to fit within and pivot about a corresponding recess or valley 342 in the actuator priming element 340. That is, the gating element 336 includes the “male” element and the priming element 340 includes the “female” element, with the male element rotating/pivoting relative to the female element. This contrasts with the actuator 132a and the tip 142a (FIG. 1C), in which the actuator 132a includes the female element (the recess 137) and the actuator priming element 140 includes the male element (the tip 142a), with the female element rotating/pivoting relative to the male element. Without intending to be bound by theory, in embodiments in which the gating element 336 includes the “male” element (e.g., the projection 337), the gating element 336 may have a greater range of rotational motion than in embodiments in which the gating element 336 includes the “female” element (e.g., the recess 137, shown in FIG. 1C).

Moreover, as also shown in FIG. 3, the projection 337 is narrower than the recess 342 such that side walls 337a of the projection 337 are spaced apart from corresponding side walls 342a of the recess 342. As a result, a gap G is formed between side walls 337a of the projection 337 and side walls 342a of the recess 342. This enables the gating element 336 to pivot, rotate, or otherwise move in response to actuation of the first actuation element 338a or the second actuation element 338b, as described above with reference to FIGS. 2B-2D. The size of the gap G, which is based at least in part on the difference in width between the projection 337 and the recess 342, can be designed based on a desired range of rotational/pivotal motion of the gating element 336. As one skilled in the art will appreciate from the disclosure herein, the actuator 332 and the priming element 340 can be used with the system 100 (FIGS. 1A-1C) in lieu of the actuator 132a and the priming element 140, and/or with another adjustable shunting system.

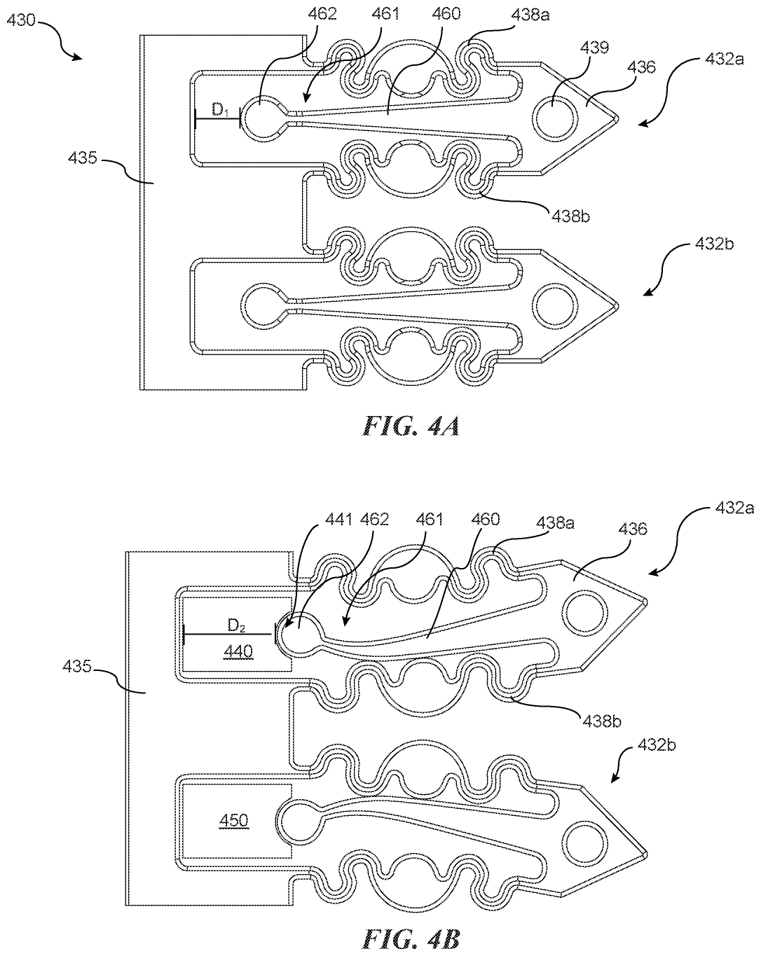

FIGS. 4A and 4B illustrate an actuator assembly 430 having a first actuator 432a and a second actuator 432b configured in accordance with select embodiments of the present technology. The first actuator 432a and the second actuator 432b can be generally similar to the actuator 132 and the actuator 332 described previously with reference to FIGS. 1A-3. For example, the first actuator 432a can include a gating element 436, an anchor element 435, a first actuation element 438a extending between the gating element 436 and the anchor element 435, and a second actuation element 438b also extending between the gating element 436 and the anchor element 435. The second actuator 432b can likewise include similar features, and can in some embodiments be the same as, or substantially the same as, the first actuator 432a. Accordingly, the following description focuses on the features of the first actuator 432a that substantially differ from the actuator 132a of FIGS. 1A-2D and/or the actuator 332 of FIG. 3. One skilled in the art will appreciate that the following description of the first actuator 432a can apply equally to the second actuator 432b.

FIG. 4A illustrates the first actuator 432a and the second actuator 432b in their “as-fabricated” or “manufactured” state, before they have been deformed (e.g., before they have been loaded onto a corresponding priming element). As shown, and unlike the actuators described with reference to FIGS. 1A-3, the first actuator 432a includes an elongated structure 460 (e.g., a projection, lever, arm, connector, etc.) that extends from the gating element 436 toward, but not all the way to, the anchor element 435. The elongated structure 460 terminates in a rotatable engagement element 462, which can have a circle, oval, or other suitably shaped profile. In the illustrated as-fabricated state, the rotatable engagement element 462 can be spaced apart from the anchor element 435 by a first distance D₁.

In some embodiments, the elongated structure 460 can be at least partially tapered such that a first width or thickness at a portion proximal the gating element 436 is greater than a corresponding second width or thickness at a portion distal the gating element 435. As a result, the elongated structure 460 includes a thinned portion 461 adjacent the rotatable engagement element 462. As described in detail below, this is expected to improve the range, repeatability and consistency of motion of gating element 436 when the first actuator 432a is actuated.

In some embodiments, the gating element 436 can optionally include an aperture 439 configured to receive a sealing element (e.g., a silicone or glass sphere; not shown) to improve sealing at a corresponding fluid flow port (not shown). Additional details regarding use of sealing elements are described in International Patent Application Publication No. WO 2022/220861 and U.S. Provisional Patent Application Nos. 63/338,393 and 63/421.851, the disclosures of which are incorporated by reference herein in their entireties.

FIG. 4B illustrates the first actuator 432a and the second actuator 432b loaded onto a first priming element 440 and a second priming element 450, respectively. As shown, the first priming element 440) extends between the anchor element 435 and the rotatable engagement element 462. In some embodiments, the priming element 440 can include a recess or notch 441 for rotatably receiving the rotatable engagement element 462. The priming element 440 can be dimensioned such that, after the first actuator 432a is loaded onto the priming element 440, the rotatable engagement element 462 is separated from the anchor element 435 by a second distance D₂ that is greater than the first distance D₁ (FIG. 4A). That is, loading the first actuator 432a onto the priming element 440 at least partially deforms the first actuator 432a relative to its as-fabricated configuration. In particular, the first actuation element 438a and the second actuation element are both stretched/lengthened relative to their preferred geometries in response to the first actuator 432a being loaded onto the priming element 440. As a result, strain is induced in the first actuation element 438a and the second actuation element 438b, which can be utilized to drive actuation of the first actuation element 438a and the second actuation element 438b as previously described. It will be appreciated that in some embodiments the priming element 440 may be shaped and sized such that, when the rotatable engagement element 462 is received therein, the rotatable engagement element 462 is separated from the anchor element 435 by a distance that is less than the first distance D₁ (that is, the first and second actuation elements 438 are shortened/compressed relative to their preferred geometries).

The first actuator 432a can operate in a similar manner as described previously for the actuator 132 in FIGS. 2A-2D. For example, the first actuation element 438a can be selectively actuated to move the gating element 436 to and/or toward a first position in which the gating element 436 is configured to impart a first fluid resistance through a corresponding fluid flow port (not shown), and the second actuation element 438b can be selectively actuated to move the gating element 436 to and/or toward a second position in which the gating element 436 is configured to impart a second fluid resistance through the corresponding fluid flow port. For purposes of illustration, FIG. 4B illustrates the first actuator 432a in the first position and the second actuator 432b in the second position, although one skilled in the art will appreciate that both the first actuator 432a and the second actuator 432b can independently move between the first position and the second position. As the first actuator 432a moves between the first position and the second position, the rotatable engagement element 462 can rotate, pivot, or otherwise move within the corresponding recess 441 of the priming element 440), similar to how the projection 337 of the actuator 332 pivots within the recess 342 during actuation of the actuator 332 described with reference to FIG. 3.

However, relative to the actuator 132a of FIGS. 1A-2D and the actuator 332 of FIG. 3, the first actuator 432a and the second actuator 432b of FIGS. 4A and 4B can be “bistable” in that they can each be selectively transitionable between two relatively low energy, or “stable,” configurations. In some embodiments, for example, the first and second low energy configurations can correspond to the configurations of the first actuator 432a that causes the gating element 436 to be in the first position and the second position. For example, in the illustrated embodiment, the first actuator 432a is shown in a first relatively low energy configuration (and thus the gating element 436 occupies the first position), while the second actuator 432b is shown in the second relatively low energy configuration (and thus the gating element occupies the second position). Of course, as described above, the first actuator 432a and the second actuator 432b can each have two relatively low energy configurations, and thus can each be independently and selectively transitionable between their two low energy configurations.

The two relatively low energy configurations are created by virtue of the elongated structure 460. For example, in both the first and second relatively low energy configurations, the thinned portion 461 of the elongated structure 460 is at least partially bent or curved to accommodate the corresponding positioning of the gating element 436. When curved, the thinned portion 461 has a relatively lower level of strain. However, to move between the first and second relatively low energy configurations (e.g., to move the gating element between the first position and the second position), the thinned portion 461 must transition through one or more intermediate shapes/configurations (e.g., straight, irregular shaped, etc.) with a relatively greater amount of strain. As a result, the first actuator 432a is unlikely to inadvertently or spontaneously transition between the first relatively low energy configuration and the second relatively low energy configuration because doing so requires sufficient energy input to drive the thinned portion 461 through the higher energy intermediate shapes. Rather, it is expected that the first actuator 432a is likely to move between the low energy states only in response to an intentional application of external energy (e.g., by actuating one of the actuation elements 438 using laser energy delivered via a laser source external to the patient). In this way, the two relatively low energy configurations are expected to be relatively more stable, which in turn is expected to make the relative position of the gating element 436 more stable.

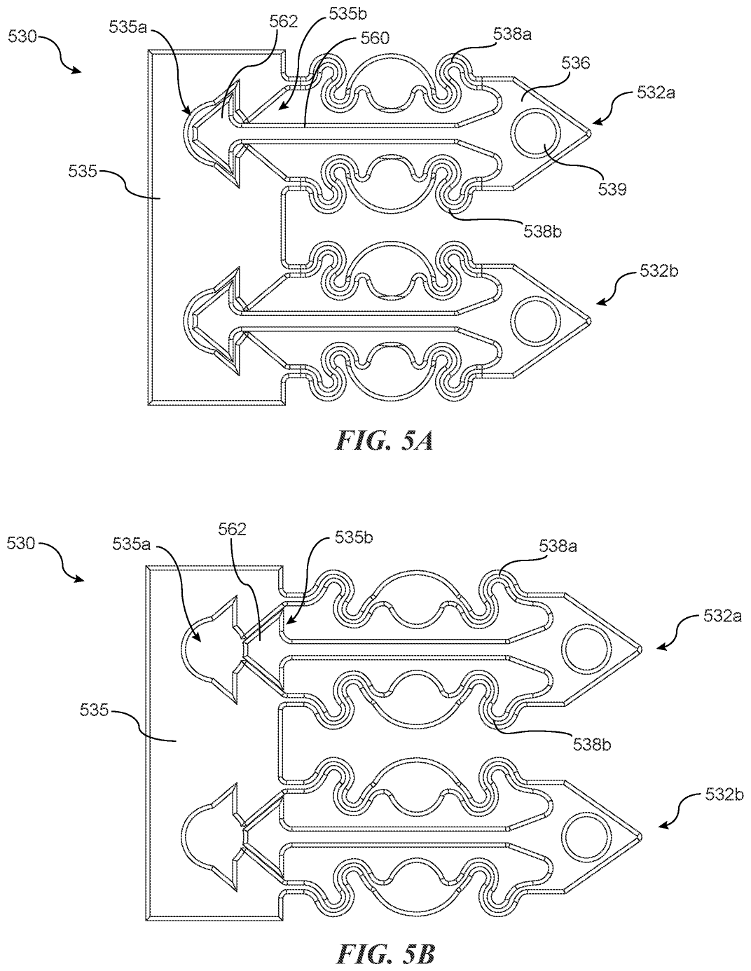

FIGS. 5A and 5B illustrate another actuator assembly 530 having a first actuator 532a and a second actuator 532b configured in accordance with select embodiments of the present technology. The first actuator 532a and the second actuator 532b can be generally similar to the first actuator 432a and the second actuator 432b described with reference to FIGS. 4A and 4B. For example, the first actuator 532a can include a gating element 536, an anchor element 535, a first actuation element 538a extending between the gating element 536 and the anchor element 535, a second actuation element 538b also extending between the gating element 536 and the anchor element 535, and an elongated structure 560 extending from the gating element 536 toward the anchor element 535. The gating element 536 can include a sealing element 539 (e.g., a glass or silicone sphere) for improving sealing at a corresponding fluid port. The second actuator 532b can likewise include similar features, and can in some embodiments be the same as, or substantially the same as, the first actuator 532a.

Relative to the actuator assembly 430 described with reference to FIGS. 4A and 4B and the other actuators described previously, the actuator assembly 530 is configured to be primed or loaded without use of a separate priming element as described in detail below. FIG. 5A illustrates the first actuator 532a and the second actuator 532b in their “as-fabricated” or “manufactured” state, and FIG. 5B illustrates the first actuator 532a and the second actuator 532b in the “primed” or “loaded” state. As shown, the elongated structure 560 includes an engagement element 562 at an end opposite the gating element 536. In the as-fabricated state as shown in FIG. 5A, the engagement element 562 can be positioned within a corresponding first well 535a formed within the anchor element 535. Of note, the engagement element 562 is not directly connected to the portion of the anchor element 535 that forms the well 535a (e.g., there is a discontinuity between surfaces forming the well 535a and a surface of the engagement element 562). To load/prime the first actuator 532a, the engagement element 562 can simply be moved from within the well 535a to a recess 535b also formed by another location of the anchor element 535, as shown in FIG. 5B. Moving the engagement element 562 from the well 535a to the recess 535b lengthens/stretches the first actuation element 538a and the second actuation element 538b, which induces strain in the first actuation element 538a and the second actuation element 538b and primes them for operation. The first actuator 532a can then operate in a manner substantially similar to the operation of the other actuators described herein to selectively move the gating element 536 between first and second positions.

The actuators described herein are expected to be advantageous for several reasons. First, as described above, the actuators can be relatively compact by virtue of the configuration of their components, which in turn enables the overall shunting system to have a smaller footprint. Depending on the application of the system, this is expected to be advantageous because systems with smaller footprints tend to induce fewer side effects, such as reduced tissue irritation, less blockage of vision, and the like. Second, in some embodiments the “pivot point” for the gating element is relatively close to the portion of the gating element that interfaces with the port (e.g., in FIG. 1C, the deepest portion 137a of the gating element 136 and the blocking portion 136a of the gating element 136 that interfaces with the first port 121 are proximate each other, such as within 50 microns, within 40 microns, within 30 microns, etc, of one another). Without being bound by theory, this may produce more controllable and consistent movement of, and/or more visible movement of, the gating element during operation, compared to embodiments in which the portion of the gating element that interfaces with the port is spaced apart from the pivot point by a greater distance. In other embodiments, the “pivot point” for the gating element is spaced apart to the portion of the gating element that interfaces with the port (e.g., in FIGS. 4B and 5B, the gating elements 436, 536 are spaced apart from the engagement elements 462, 562 by the elongated structure). Without being bound by theory, this may produce a more stable actuator due to creation of two relatively low energy configurations separated by one or more intermediate higher energy configurations.

Although the present actuators are described in the context of the system 100 (FIGS. 1A-1C), the actuators described herein can be implanted with other shunts and systems to selectively control the flow of fluid. For example, the actuators described herein can be used with the shunting elements and systems described in U.S. Patent Application Publication No. US2021/0251806, International Patent Application No. PCT/US21/49140, and U.S. Provisional Patent Application No. 63/292,164, each of which is incorporated by reference herein in its entirety.

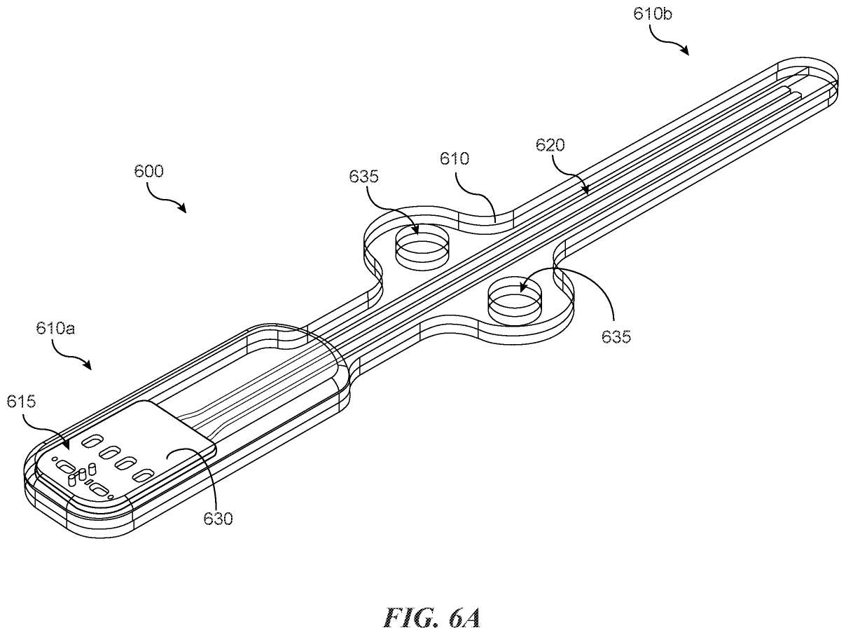

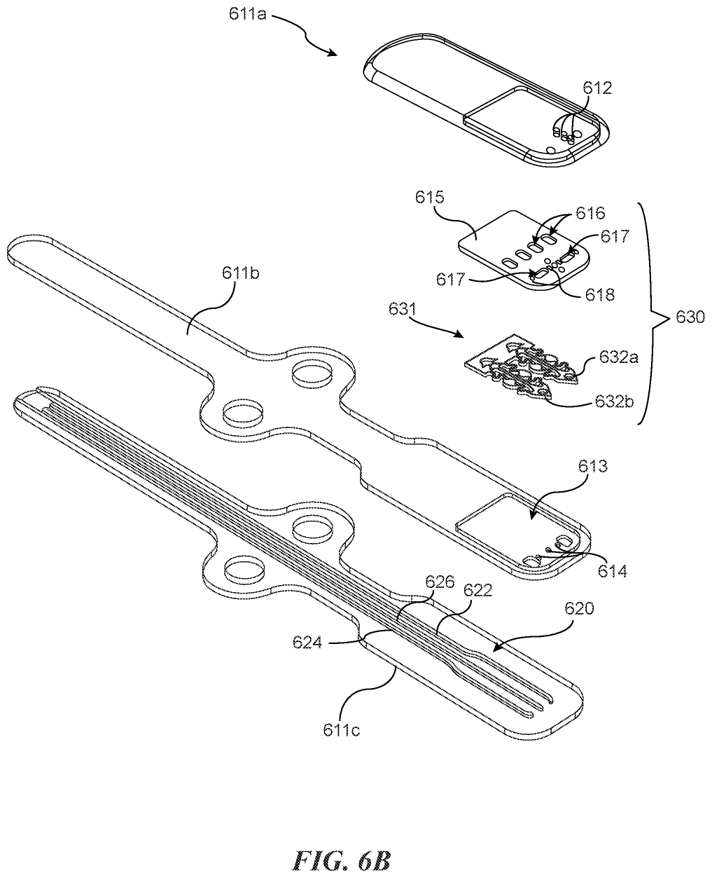

FIGS. 6A and 6B show another variation of an adjustable shunting system 600 that can include any of the actuators described herein and is configured in accordance with select embodiments of the present technology. More specifically, FIG. 6A is a perspective view of the system 600, and FIG. 6B is an exploded view of the system 600. Referring first to FIG. 6A, the system 600 can be generally similar to the system 100 described with reference to FIGS. 1A-1C. For example, the system 600 can include an elongated housing or shunting element 610 composed of a biocompatible and at least partially flexible material, and a flow control cartridge 630. The shunting element 610 can include a fluid resistor network 620 extending between a first end portion 610a of the shunting element 610 and a second end portion 61b of the shunting element 610. Similar to the fluid resistor network 120 described with reference to FIGS. 1A-1C, the fluid resistor network 620 permits fluid to drain through the shunting element 610. The shunting element 610 can also include one or more apertures 635 extending therethrough that can be used to suture the system 600 to patient tissue at a target implant location.

Referring to FIG. 6B, the shunting element 610 can comprise a plurality of layers that can be stacked atop one another when the system 600 is assembled. For example, in the illustrated embodiment the shunting element 610 includes a first layer 611a, a second layer 611b, and a third layer 611c. The first layer 611a is configured to form a cover over the flow control cartridge 630 when the system 600 is assembled. The first layer 611a can also include a plurality of fluid inlets or apertures 612 for permitting fluid to flow into the system 600. The second layer 611b can include a cavity or chamber 613 sized and shaped to receive the flow control cartridge 630. The second layer 611b can also include one or more fluid flow ports 614 for permitting fluid flowing into the system 600 via the fluid inlets 612 in the first layer 611a to drain into the fluid resistor network 620. The third layer 611c can include the fluid resistor network 620, which in the illustrated embodiment comprises a first channel 622, a second channel 624, and a third channel 626, although in other embodiment can comprise more or fewer channels. The void space of the channels 622, 624, 626 are formed within the third layer 611c, but one side of the channels (e.g., the “top” of the channels) is formed via a lower surface of the second layer 611b. Additional details regarding shunting systems comprising multiple layers are described in International Patent Application Publication No. WO 2023/004067, the disclosure of which is incorporated by reference herein in its entirety.

The flow control cartridge 630 can include an actuator assembly 631 comprising a first actuator 632a and a second actuator 632b, and a plate 615 that forms a “backbone” for the actuator assembly 631. The actuator assembly and the actuators 632a, 632b can be generally similar to or the same as any of the actuator assemblies and actuators described throughout this Detailed Description. The plate 615, which can be formed of a generally rigid material such as superelastic Nitinol or other suitable metals, alloys, glass, or plastic, can include any priming or bracing element (e.g., for deforming and loading the actuators as described above with reference to FIGS. 1C-4B). Of note, as shown in both FIGS. 6A and 6B, the flow control cartridge 630 in the system 600 has a different orientation relative to the flow control cartridge 130 of the system 100 of FIGS. 1A-1C. In particular, the plate 615 is oriented “above” the actuator assembly 631 when the system 600 is assembled. As a result, the plate 615 includes a plurality of openings 616 that are positioned to align with corresponding targets on the first actuator 632a and the second actuator 632b, e.g., to permit energy such as laser energy to be directed to the first actuator 632a and the second actuator 632b to drive actuation thereof. The plate 615 also includes a plurality of fluid openings 617 that permit fluid to flow from the fluid inlets 612 in the first layer 611a and through the corresponding fluid ports 614 in the second layer 611b. Because the plate 615 sits above, and therefore at least partially blocks the view of, the actuator assembly 631, the plate 615 can further include one or more system state indicators 618 to provide (a) feedback to the user on the relative position of the first actuator 632a and the second actuator 632b, and/or (b) instructions for where to direct energy to change a position of the first actuator 632a and/or the second actuator 632b, e.g., to change a fluid resistance through the system 600. Additional features of system state indicators that can be used with adjustable shunting systems are described in U.S. Provisional Patent Application No. 63/481,955, the disclosure of which is incorporated by reference herein in its entirety.

### EXAMPLES

Several aspects of the present technology are set forth in the following examples:
1. An implantable system for shunting fluid between a first body region and a second body region of a patient, the system comprising;
a shunting element having a channel extending therethrough and a port in fluid communication with the channel;
a shape memory actuator configured to control flow of fluid through the port, the shape memory actuator comprising
   an anchor element;
   a gating element configured to moveably interface with the port,
   a first actuation element extending between the anchor element and the gating element, and
   a second actuation element extending between the anchor element and the gating element,
   wherein the first actuation element, when actuated, is configured to pivot the gating element in a first direction relative to the port, and
   wherein the second actuation element, when actuated, is configured to pivot the gating element in a second direction relative to the port, the second direction being opposite the first direction; and
an actuator priming element configured to receive the shape memory actuator, wherein the actuator priming element extends between the anchor element and the gating element and is shaped and sized to deform the shape memory actuator relative to its preferred geometry when the shape memory actuator is coupled thereto.
2. The system of example 1 wherein:
the actuator priming element includes a first end portion at the gating element and a second end portion at the anchor element; and
the gating element is configured to pivot about the first end portion of the actuator priming element when moving in the first direction and the second direction.
3. The system of example 1 or example 2 wherein the gating element includes a recess, and wherein the actuator priming element has a tapered end portion configured to extend into the recess.
4. The system of example 3 wherein the gating element is configured to pivot about the tapered end portion of the actuator priming element when moving in the first direction and the second direction.
5. The system of example 3 wherein the tapered end portion of the actuator priming element contacts the gating element at a bottom of the recess, and wherein sides of the tapered end portion are spaced apart from corresponding sides of the recess by a gap.
6. The system of example 1 or example 2 wherein the gating element includes a projection, and wherein the actuator priming element has a recess configured to receive the projection.
7. The system of example 6 wherein the projection element is configured to pivot relative to the recess as the gating element moves in the first direction and the second direction.
8. The system of example 6 wherein side walls of the projection are spaced apart from side walls of the recess.
9 The system of claim 1 wherein the shape memory actuator has a width of less than about 100 microns.
10. The system of any of examples 1-9 wherein the shape memory actuator has a width of less than about 50 microns.
11. The system of any of examples 1-10 wherein the shape memory actuator is a single, unitary component composed of Nitinol.
12. The system of any of examples 1-11 wherein the shunting system is an intraocular shunting system configured to be implanted in an eye of the patient.
13. A shape memory actuator for use with an implantable shunting system for treating a patient, the shape memory actuator comprising:
a gating element configured to moveably interface with a fluid port on the implantable shunting system when the shape memory actuator is operably coupled to the implantable shunting system;
a first actuation element coupled to the gating element,
   wherein, when actuated the first actuation element is configured to pivot the gating element in a first direction;
a second actuation element coupled to the gating element,
   wherein, when actuated the second actuation element is configured to pivot the gating element in a second direction; and
an elongated projection coupled to the gating element and extending between the first actuation element and the second actuation element.
14. The shape memory actuator of example 13 wherein the elongated projection includes a thinned portion.
15. The shape memory actuator of example 13 or example 14, further comprising an engagement element connected to the elongated structure at an end opposite of the gating element, wherein the gating element and the elongated structure are configured to pivot about the engagement element.
16. The shape memory actuator of example 15, further comprising an anchor portion, wherein the first actuation element and the second actuation element extend between the anchor portion and the gating element.
17. The shape memory actuator of example 16 wherein:
the engagement element is moveable between a first position relative to the anchor portion and a second position relative to the anchor position different than the first position; and
the shape memory actuator is configured such that moving the engagement element from the first position to the second position increases an amount of strain in the first actuation element and the second actuation element.
18. The shape memory actuator of example 17 wherein the shape memory actuator is configured such that moving the engagement element from the first position to the second position compresses the first actuation element and the second actuation element.
19. The shape memory actuator of example 17 wherein the shape memory actuator is configured such that moving the engagement element from the first position to the second position stretches the first actuation element and the second actuation element
20. The shape memory actuator of any of examples 13-19 wherein, in response to the first actuation element or the second actuation element being actuated:
the shape memory actuator is configured to transition between (a) a first relatively low energy state in which the gating element occupies a first position and (b) a second relatively low energy state in which the gating element occupies a second position different than the first position, and
the shape memory actuator is configured to transition through one or more relatively higher energy states as it moves from the first relatively low energy state to the second relatively low energy state.
21. The shape memory actuator of example 20 wherein the elongated projection is configured to change shape as the shape memory actuator transitions between the first relatively low energy state and the second relatively low energy state.
22. The shape memory actuator of example 20 wherein the elongated projection includes a thinned portion, and wherein the elongated structure is configured to bend at the thinned portion when the shape memory actuator is in the first relatively low energy state and the second relatively low energy state.
23. An implantable shunting element having a channel extending therethrough for shunting fluid between a first body region and a second body region of a patient system, the shunting element comprising:
an anchor element;
a gating element configured to moveably interface with a port in fluid communication with the channel;
an actuator priming element extending between the anchor element and the gating element, wherein the actuator priming element includes (a) a first end portion at the gating element and (b) a second portion at the anchor element;
a first shape memory actuation element extending between the anchor element and the gating element, wherein the first actuation element, when actuated, is configured to pivotably move the gating element in a first direction; and
a second shape memory actuation element extending between the anchor element and the gating element, wherein the second actuation element, when actuated, is configured to pivotably move the gating element in a second direction opposite the first direction,
wherein, when moving in the first direction and the second direction, the gating element is configured to pivotably move about the first end portion of the actuator priming element.
24. The shunting element of example 23 wherein, when the actuator priming element is coupled between the anchor element and the gating element, the actuator priming element is shaped and sized to deform both the first shape memory actuation element and the second shape memory actuation element relative to their preferred geometries.
25. The shunting element of example 23 and example 24 wherein the gating element includes:
a blocking portion configured to moveably interface with the port; and
a pivot region about which the blocking portion is configured to pivot upon actuation of the first actuation element or the second actuation element.
26. The shunting element of example 25 wherein an interface between the first end portion of the actuator priming element and the gating element defines the pivot region.
27. The shunting element of example 25 wherein the gating element is V-shaped.
28. The shunting element of example 25 wherein the blocking portion and the pivot region are spaced apart by less than about 100 microns.
29. The shunting element of any of examples 23-28 wherein, in response to the first actuation element or the second actuation element being actuated:
the shunting element is configured to transition between (a) a first relatively low energy state in which the gating element occupies a first position and (b) a second relatively low energy state in which the gating element occupies a second position different than the first position, and
the shunting element is configured to transition through one or more relatively higher energy states as it moves from the first relatively low energy state to the second relatively low energy state.
30. The shunting element of example 29 wherein the shunting element is configured to occupy the first relatively low energy state after actuation of the first actuation element and the second relatively low energy state after actuation of the second actuation element.
31. The shunting element of any of examples 23-30 wherein the first actuation element and the second actuation element each have a width of less than about 100 microns.
32. The shunting element of any of examples 23-30 wherein the first actuation element and the second actuation element each have a width of less than about 50 microns.
33. The shunting element of any of examples 23-30 wherein the anchor element, the gating element, the first actuation element, and the second actuation element comprise a single, unitary structure.
34. The shunting element of any of examples 23-30 wherein each of the anchor element, the gating element, the first actuation element, and the second actuation element are composed of the same material.
35. The shunting element of example 34 wherein the material is Nitinol.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone. B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. An implantable system for shunting fluid between a first body region and a second body region of a patient, the system comprising:
a shunting element having a channel extending therethrough and a port in fluid communication with the channel;
a shape memory actuator configured to control flow of fluid through the port, the shape memory actuator comprising
an anchor element;
a gating element configured to moveably interface with the port,
a first actuation element extending between the anchor element and the gating element, and
a second actuation element extending between the anchor element and the gating element,
wherein the first actuation element, when actuated, is configured to pivot the gating element in a first direction relative to the port, and
wherein the second actuation element, when actuated, is configured to pivot the gating element in a second direction relative to the port, the second direction being opposite the first direction; and
an actuator priming element configured to receive the shape memory actuator, wherein the actuator priming element extends between the anchor element and the gating element and is shaped and sized to deform the shape memory actuator relative to its preferred geometry when the shape memory actuator is coupled thereto.

2. The system of claim 1 wherein:
the actuator priming element includes a first end portion at the gating element and a second end portion at the anchor element; and
the gating element is configured to pivot about the first end portion of the actuator priming element when moving in the first direction and the second direction.

3. The system of claim 1 wherein the gating element includes a recess, and wherein the actuator priming element has a tapered end portion configured to extend into the recess.

4. The system of claim 3 wherein the gating element is configured to pivot about the tapered end portion of the actuator priming element when moving in the first direction and the second direction.

5. The system of claim 3 wherein the tapered end portion of the actuator priming element contacts the gating element at a bottom of the recess, and wherein sides of the tapered end portion are spaced apart from corresponding sides of the recess by a gap.

6. The system of claim 1 wherein the gating element includes a projection, and wherein the actuator priming element has a recess configured to receive the projection.

7. The system of claim 6 wherein the projection element is configured to pivot relative to the recess as the gating element moves in the first direction and the second direction.

8. The system of claim 6 wherein side walls of the projection are spaced apart from side walls of the recess.

9. The system of claim 1 wherein the shape memory actuator has a width of less than about 100 microns.

10. The system of claim 1 wherein the shape memory actuator has a width of less than about 50 microns.

11. The system of claim 1 wherein the shape memory actuator is a single, unitary component composed of Nitinol.

12. The system of claim 1 wherein the shunting system is an intraocular shunting system configured to be implanted in an eye of the patient.

13. A shape memory actuator for use with an implantable shunting system for treating a patient, the shape memory actuator comprising:
a gating element configured to moveably interface with a fluid port on the implantable shunting system when the shape memory actuator is operably coupled to the implantable shunting system;
a first actuation element coupled to the gating element,
wherein, when actuated the first actuation element is configured to pivot the gating element in a first direction;
a second actuation element coupled to the gating element,
wherein, when actuated the second actuation element is configured to pivot the gating element in a second direction; and
an elongated projection coupled to the gating element and extending between the first actuation element and the second actuation element.

14. The shape memory actuator of claim 13 wherein the elongated projection includes a thinned portion.

15. The shape memory actuator of claim 13, further comprising an engagement element connected to the elongated structure at an end opposite of the gating element, wherein the gating element and the elongated structure are configured to pivot about the engagement element.

16. The shape memory actuator of claim 15, further comprising an anchor portion, wherein the first actuation element and the second actuation element extend between the anchor portion and the gating element.

17. The shape memory actuator of claim 16 wherein:
the engagement element is moveable between a first position relative to the anchor portion and a second position relative to the anchor position different than the first position; and
the shape memory actuator is configured such that moving the engagement element from the first position to the second position increases an amount of strain in the first actuation element and the second actuation element.

18. The shape memory actuator of claim 17 wherein the shape memory actuator is configured such that moving the engagement element from the first position to the second position compresses the first actuation element and the second actuation element.

19. The shape memory actuator of claim 17 wherein the shape memory actuator is configured such that moving the engagement element from the first position to the second position stretches the first actuation element and the second actuation element

20. The shape memory actuator of claim 13 wherein, in response to the first actuation element or the second actuation element being actuated:
the shape memory actuator is configured to transition between (a) a first relatively low energy state in which the gating element occupies a first position and (b) a second relatively low energy state in which the gating element occupies a second position different than the first position, and
the shape memory actuator is configured to transition through one or more relatively higher energy states as it moves from the first relatively low energy state to the second relatively low energy state.

21. The shape memory actuator of claim 20 wherein the elongated projection is configured to change shape as the shape memory actuator transitions between the first relatively low energy state and the second relatively low energy state.

22. The shape memory actuator of claim 20 wherein the elongated projection includes a thinned portion, and wherein the elongated structure is configured to bend at the thinned portion when the shape memory actuator is in the first relatively low energy state and the second relatively low energy state.

23. An implantable shunting element having a channel extending therethrough for shunting fluid between a first body region and a second body region of a patient system, the shunting element comprising:
an anchor element;
a gating element configured to moveably interface with a port in fluid communication with the channel;
an actuator priming element extending between the anchor element and the gating element, wherein the actuator priming element includes (a) a first end portion at the gating element and (b) a second portion at the anchor element;
a first shape memory actuation element extending between the anchor element and the gating element, wherein the first actuation element, when actuated, is configured to pivotably move the gating element in a first direction; and
a second shape memory actuation element extending between the anchor element and the gating element, wherein the second actuation element, when actuated, is configured to pivotably move the gating element in a second direction opposite the first direction,
wherein, when moving in the first direction and the second direction, the gating element is configured to pivotably move about the first end portion of the actuator priming element.

24. The shunting element of claim 23 wherein, when the actuator priming element is coupled between the anchor element and the gating element, the actuator priming element is shaped and sized to deform both the first shape memory actuation element and the second shape memory actuation element relative to their preferred geometries.

25. The shunting element of claim 23 wherein the gating element includes:
a blocking portion configured to moveably interface with the port; and
a pivot region about which the blocking portion is configured to pivot upon actuation of the first actuation element or the second actuation element.

26. The shunting element of claim 25 wherein an interface between the first end portion of the actuator priming element and the gating element defines the pivot region.

27. The shunting element of claim 25 wherein the gating element is V-shaped.

28. The shunting element of claim 25 wherein the blocking portion and the pivot region are spaced apart by less than about 100 microns.

29. The shunting element of claim 23 wherein, in response to the first actuation element or the second actuation element being actuated:
the shunting element is configured to transition between (a) a first relatively low energy state in which the gating element occupies a first position and (b) a second relatively low energy state in which the gating element occupies a second position different than the first position, and
the shunting element is configured to transition through one or more relatively higher energy states as it moves from the first relatively low energy state to the second relatively low energy state.

30. The shunting element of claim 29 wherein the shunting element is configured to occupy the first relatively low energy state after actuation of the first actuation element and the second relatively low energy state after actuation of the second actuation element.

31. The shunting element of claim 23 wherein the first actuation element and the second actuation element each have a width of less than about 100 microns.

32. The shunting element of claim 23 wherein the first actuation element and the second actuation element each have a width of less than about 50 microns.

33. The shunting element of claim 23 wherein the anchor element, the gating element, the first actuation element, and the second actuation element comprise a single, unitary structure.

34. The shunting element of claim 23 wherein each of the anchor element, the gating element, the first actuation element, and the second actuation element are composed of the same material.

35. The shunting element of claim 34 wherein the material is Nitinol.
